(19) United States
(12) Patent Application Publication
(10) Pub. No.: US 2021/0280277 A1
(43) Pub. Date: Sep. 9, 2021

(54) MATERIAL DESIGN PROGRAM

(71) Applicant: KABUSHIKI KAISHA TOYOTA CHUO KENKYUSHO, Nagakute-shi (JP)

(72) Inventors: Kenroh MATSUDA, Nagoya-shi (JP); Seiji KAJITA, Nagakute-shi (JP)

(73) Assignee: KABUSHIKI KAISHA TOYOTA CHUO KENKYUSHO, Nagakute-shi (JP)

(21) Appl. No.: 17/195,210

(22) Filed: Mar. 8, 2021

(30) Foreign Application Priority Data
Mar. 9, 2020 (JP) 2020-040348

(51) Int. Cl. G16C 20/50; G06F 30/27; G16C 20/70

(52) U.S. Cl. CPC G16C 20/50; G06F 2111/18; G16C 20/70; G06F 30/27

(57) ABSTRACT

The material design program makes a computer perform: (A) Procedure A of displaying a molecular structure of an organic compound (A) or a fragment as a starting point in a VR space; (B) Procedure B of, when modifying the molecular structure and creating a new organic compound (B) in the VR space, displaying a molecular structure of the organic compound (B) in the VR space; (C) Procedure C of creating an explanatory variable (X_{B}) that may correlate with an objective variable (Y_{B}), which is a physical property value of the organic compound (B), based on the molecular structure of the organic compound (B); (D) Procedure D of estimating the objective variable (Y_{B}) of an organic compound (B) by using a machine learning model; and (E) Procedure E of, when selecting a new modification of the organic compound (B), repeating Procedure B to Procedure D.

### FIELD OF THE INVENTION

The present invention relates to a material design program, more specifically, a material design program that can display and modify a molecular structure of an organic compound in a virtual reality (VR) space and can estimate a physical property value of an unknown organic compound created in a VR space.

### BACKGROUND OF THE INVENTION

A physical property value (for example, viscosity) of an organic compound differs depending on the molecular structure. When an organic compound is used for a certain use,

(a) a specific organic compound having a physical property value suitable for the use is selected from innumerably existing organic compounds or
(b) an organic compound having a molecular structure suitable for the use is designed.

As a method for knowing whether or not a designed organic compound is suitable for an intended use when the organic compound is designed, there are

(a) a method for estimating a physical property value of the organic compound by using simulations such as molecular dynamics (MD) calculation and ab-initio calculation and
(b) a method for examining whether or not the organic compound is suitable for the use by experiment.

When MD calculation or experiment is used, however, it takes a few days to a few weeks before whether or not a designed organic compound is suitable for an intended use is determined in many cases. A problem of those methods therefore is that a designer's thinking is temporarily stopped.

In a VR space in contrast, a molecular structure of an organic compound can be modified relatively easily. Further, a user with specialized knowledge can also predict the physical property value of an organic compound to some extent from a displayed molecular structure. Various proposals therefore have been made so far regarding the display of an object in a VR space.

Patent Literature 1 for example discloses a system for tracking trainee proficiency when a trainee learns a welding method in a real-time simulated virtual reality welding training environment although it does not intend to create a new organic compound.

The literature describes that:

(A) when the system is used, it is possible to simulate a welding pool having real-time molten metal fluidity and heat dissipation characteristics in a virtual realty space;

(B) when an imitation welding tool is displayed in a virtual reality space, it is possible for a user to adjust welding technique in real-time by giving real-time visual feedback to the user; and

(C) this makes it possible to assist a user in learning welding technique.

Non-patent Literature 1 discloses a VR platform focused on collaboration by visualization, operation, simulation, and communication of molecular structure data.

The VR platform described in the literature is developed with the aim of minimizing the necessity of technological know-how for using applications and expressing a molecular structure and dynamics more intuitively, and all participants can equally investigate, modify, and operate a molecular structure. This VR platform makes it possible to incorporate opinions from experts in real-time in not only drug discovery but also various fields.

The method described in Non-patent Literature 1 aims to visualize a molecular structure of an organic compound and does not indicate a physical property value of an organic compound. In order to know a physical property value of a visualized organic compound therefore, another method (for example, MD calculation or experiment) has to be used.

Meanwhile, it is also conceivable to search an organic compound having a target physical property value by using a molecular structure search of a reinforced learning task. A problem in the molecular structure search of the reinforced learning task, however, is that a large number of high-performance but hardly-synthesizable molecules are created. This is because it is difficult to estimate factors such as □ease of synthesis□ and □cost□, which are hardly converted into data.

### CITATION LIST

### Patent Literature

[Patent Literature 1] Japanese Unexamined Patent Application Publication No. 2019-164374

### Non-Patent Literature

[Non-patent Literature 1] L. J. Kingsley et al., Journal of Molecular Graphics and Modelling 89(2019) 234-241

### SUMMARY OF THE INVENTION

A problem to be solved by the present invention is to provide a material design program that can display and modify a molecular structure of an organic compound in a VR space and can estimate a physical property value of an unknown organic compound created in a VR space.

In order to solve the above problem, a material design program according to the present invention makes a computer perform the following procedures:

(A) Procedure A of requesting an operator to input a molecular structure of an organic compound (A) or a fragment as a starting point, displaying a stereoscopic image of the inputted molecular structure in a VR space, and simultaneously storing the inputted molecular structure in a memory;

(B) Procedure B of, when the operator modifies the molecular structure of the organic compound (A) or the fragment and creates a new organic compound (B) in the VR space, displaying a stereoscopic image of a molecular structure of the organic compound (B) in the VR space and simultaneously storing the molecular structure of the organic compound (B) in the memory;

(C) Procedure C of creating an explanatory variable (X_{B}) that may correlate with an objective variable (Y_{B}), which is a physical property value of the organic compound (B), based on the molecular structure of the organic compound (B) and storing the explanatory variable (X_{B}) in the memory;

(D) Procedure D of estimating the objective variable (Y_{B}) from the explanatory variable (X_{B}) by using a machine learning model in which the relationship between an objective variable (Y_{C}) of an organic compound (C) the molecular structure and physical property value of which are known and an explanatory variable (X_{C}) that may correlate with the objective variable (Y_{C}) has been learned beforehand, displaying the estimated objective variable (Y_{B}) in the VR space, and simultaneously storing the objective variable (Y_{B}) in the memory; and

(E) Procedure E of repeating Procedure B to Procedure D when the operator selects a new modification of the organic compound (B) after Procedure D.

When a new organic compound (B) is created by modifying a molecular structure in a VR space, an explanatory variable (for example, a type and a number of atoms or a bonding state of atoms) that correlates with an objective variable (for example, viscosity) of the organic compound (B) is created. Successively, the objective variable is estimated from the created explanatory variable by using a previously learned machine learning model. Further, the estimated objective variable together with the molecular structure is displayed in the VR space.

When such operations are repeated in the VR space, it is possible to search a new organic compound having a physical property value suitable for an intended use. Further, when an operator is a person who has specialized knowledge on synthesis of an organic compound, it is possible to modify a molecular structure in consideration of factors such as □ease of synthesis□ and □cost□, which are hardly converted into data.

Further, since a molecular structure and a physical property value are displayed simultaneously, a material designer is easy to get inspiration on a novel material. Furthermore, since simultaneous visualization by a large number of people including participants in remote areas is possible, it is possible to give inspiration on a novel material also to collective intelligence.

### BRIEF DESCRIPTION OF THE DRAWINGS

FIG. 1A is a flowchart of a material design program according to the present invention;

FIG. 1B is a continuation of FIG. 1A;

FIG. 2A is a view showing a process of displaying a molecular structure and a physical property value as a starting point;

FIG. 2B is a view showing a process of bonding a new fragment to a molecule;

FIG. 3A is a view showing a process of replacing an atom (for example, an oxygen atom) in a molecule with another atom (for example, a carbon atom);

FIG. 3B is a view showing a process of eliminating an atom in a molecule;

FIG. 4A is a view showing a process of changing an intermolecular bond (for example, a double bond) to another bond (for example, a single bond);

FIG. 4B is a view showing a process of cleaving a long chain molecule in the middle;

FIG. 5A is a view showing a process of recombining two cleaved molecules; and

FIG. 5B is a view showing a process of applying a material design program according to the present invention to multi-person discussion.

### DETAILED DESCRIPTION OF THE PREFERRED EMBODIMENTS

An embodiment according to the present invention is explained hereunder in detail.

### [1. Material Design Program]

A material design program according to the present invention makes a computer perform the following procedures:

(A) Procedure A of requesting an operator to input a molecular structure of an organic compound (A) or a fragment as a starting point, displaying a stereoscopic image of the inputted molecular structure in a VR space, and simultaneously storing the inputted molecular structure in a memory;

(B) Procedure B of, when the operator modifies the molecular structure of the organic compound (A) or the fragment and creates a new organic compound (B) in the VR space, displaying a stereoscopic image of a molecular structure of the organic compound (B) in the VR space and simultaneously storing the molecular structure of the organic compound (B) in the memory;

(C) Procedure C of creating an explanatory variable (X_{B}) that may correlate with an objective variable (Y_{B}), which is a physical property value of the organic compound (B), based on the molecular structure of the organic compound (B) and storing the explanatory variable (X_{B}) in the memory;

(D) Procedure D of estimating the objective variable (Y_{B}) from the explanatory variable (X_{B}) by using a machine learning model in which the relationship between an objective variable (Y_{C}) of an organic compound (C) the molecular structure and physical property value of which are known and an explanatory variable (X_{C}) that may correlate with the objective variable (Y_{C}) has been learned beforehand, displaying the estimated objective variable (Y_{B}) in the VR space, and simultaneously storing the objective variable (Y_{B}) in the memory; and

(E) Procedure E of repeating Procedure B to Procedure D when the operator selects a new modification of the organic compound (B) after Procedure D.

The material design program according to the present invention, when the operator does not select a new modification of the organic compound (B) in Procedure E, may further include

(F) Procedure F of performing molecular simulation of the organic compound (B) and calculating a physical property value corresponding to the objective variable (Y_{B}).

Further, the material design program according to the present invention, when a database of the molecular structures, the objective variables (Y_{C}), and the explanatory variables (X_{C}) of the organic compounds (C) used when the machine learning model is created is stored in the memory, may further include

(G) Procedure G of judging whether or not an organic compound (C) having the explanatory variable (X_{C}) that exactly matches the explanatory variable (X_{B}) of the organic compound (B) exists in the database after Procedure C, and

(H) Procedure H of, when an organic compound (C) having an exactly matching explanatory variable exists in the database, reading the objective variable (Y_{C}) of the organic compound (C) having the exactly matching explanatory variable and displaying the objective variable (Y_{C}) as the objective variable (Y_{B}) in the VR space.

[1.1. Display in VR space (Procedure A)]

Firstly, requesting an operator to input a molecular structure of an organic compound (A) or a fragment as a starting point, displaying a stereoscopic image of the inputted molecular structure in a VR space, and simultaneously storing the inputted molecular structure in a memory (Procedure A).

As the starting point, either the organic compound (A) or the fragment may be used.

□A fragment□ is referred to as a partial structure constituting an organic compound (B) the objective variable (Y_{B}) of which is estimated. Examples of the partial structure are

(a) an atom included in the organic compound (B), and

(b) anatomic group included in the organic compound (B) (for example, an N-numbered ring (N≥3), an organic compound having a lower molecular weight and/or a simpler structure than the organic compound (B), C—C, O—C, C═C, ═O, etc.).

When a fragment is selected as the starting point of molecular structure modification, the molecular structure of the fragment designated by an operator is displayed in the VR space.

Meanwhile, as will be described later, in addition to a machine learning model for estimating an objective variable, a database of molecular structures, objective variables (Y_{C}), and explanatory variables (X_{C}) of organic compounds (C) used when the machine learning model is created may be stored in a memory. On such an occasion, it is possible to select an organic compound (C) stored in the memory as the organic compound (A) of the starting point and display the molecular structure and the objective variable in the VR space.

A method for displaying a stereoscopic image of a molecular structure in a VR space is not particularly limited. Software for displaying a stereoscopic image in a VR space is commercially available and hence such software may be used. Examples of the software for displaying a stereoscopic image in a VR space are Unity, UNREAL, and ENGINE.

[1.2. Modification of molecular structure (Procedure B)]

Successively, when the operator modifies the molecular structure of the organic compound (A) or the fragment in the VR space and creates a new organic compound (B), displaying a stereoscopic image of the molecular structure of the organic compound (B) in the VR space and simultaneously storing the molecular structure of the organic compound (B) in the memory (Procedure B).

Examples of the method for modifying a molecular structure are

(a) a method through which an operator mounts a head mounted display and modifies a molecular structure using a button arranged in a VR space or a hand gesture, and

(b) a method through which an operator mounts a head mounted display and modifies a molecular structure using a controller attached to the head mounted display.

In particular, the method of modifying a molecular structure using a button or a hand gesture has the advantage of being easy to be operated intuitively because movement and molecular structure modification are easily linked. For example, when there is a button of the same mark as □Return□ in a general web browser in a VR space, the result of pressing the button can be easily imagined even without the instruction of □Molecule returns to structure before modification□. Further, a structure can be modified by easy-to-image operation like the case where, if a single bond is touched with □two□ fingers, it will be modified to a □double□ bond.

### [1.3. Creation of Explanatory Variable (Procedure C)]

Successively, creating an explanatory variable (X_{B}) that may correlate with an objective variable (Y_{B}), which is a physical property value of the organic compound (B), based on the molecular structure of the organic compound (B) and storing the explanatory variable (X_{B}) in the memory (Procedure C).

□An objective variable□ is a physical property value of an organic compound and is referred to as a variable to focus on when a molecular structure is designed. Examples of the objective variable are viscosity, a density, a diffusion coefficient, and ion conductivity.

□An explanatory variable□ is a numerical datum that expresses a characteristic of a molecular structure of an organic compound and is referred to as a variable that may correlate with an objective variable.

Examples of the numerical datum that can be an explanatory variable are the number of C atoms, the number of O atoms, the number of rings, and the number and the presence or absence of specific functional groups and partial structures.

A method of creating an explanatory variable (X_{B}) from a molecular structure of an organic compound (B) is not particularly limited. Software for creating an explanatory variable is open to the public or commercially available and hence such software may be used. Examples of the open-to-public or commercially-available software for creating an explanatory variable are RDKit, mordred, thermo, and MOE.

### [1.4. Display of Objective Variable]

Successively, displaying the objective variable (Y_{B}) of the organic compound (B) in the VR space. Examples of the method of obtaining the objective variable (Y_{B}) of the organic compound (B) are

(a) a method of estimating an objective variable by using a machine learning model, and

(b) a method of reading an objective variable corresponding to a molecular structure from a database.

### [1.4.1 Estimation of Objective Variable by Using Machine Learning Model (Procedure D)]

When the objective variable (Y_{B}) is estimated by using a machine learning model, firstly it is necessary to make the relationship between an objective variable (Y_{C}) of an organic compound (C) the molecular structure and physical property value of which are known and an explanatory variable (X_{C}) that may correlate with the objective variable (Y_{C}) learn beforehand and create the machine learning model.

In order to create the machine learning model, it is necessary to determine the molecular structure of the organic compound (C), the objective variable (Y_{C}) that is the physical property value of the organic compound (C), and the explanatory variable (X_{C}) that may correlate with the objective variable (Y_{C}). In the case of an organic compound (C) the molecular structure of which is known but the physical property value of which is unknown, either the physical property value is measured by experiment or an estimated value of the objective variable (Y_{C}) (namely, physical property value) is obtained beforehand by using a highly accurate molecular simulation method such as a molecular dynamics (MD) method, and ab-initio calculation.

The method of creating the explanatory variable (X_{C}) from the molecular structure of the organic compound (C) is the same as the method of creating the explanatory variable (X_{B}) and hence the explanation is omitted.

Successively, with regard to many organic compounds (C), a machine learning model is created by making a computer learn the relationships between objective variables (Y_{C}) and explanatory variables (X_{C}). The method of machine learning is not particularly limited. Software for machine learning is open to the public or commercially available and hence such software may be used. Examples of the software for machine learning are Python, R, and MATLAB.

Estimating the objective variable (Y_{B}) from the explanatory variable (X_{B}) by using the machine learning model obtained in this way, displaying the estimated objective variable (Y_{B}) in the VR space, and simultaneously storing the objective variable (Y_{B}) in the memory (Procedure D).

### [1.4.2. Display of Objective Variable Using Database (Procedures G and H)]

If the machine learning model is once created, it is possible to estimate the objective variable (Y_{B}) of the organic compound (B) even without a database of the molecular structures, the objective variables (Y_{C}), and the explanatory variables (X_{C}) of the organic compounds (C) used when the machine learning model is created. When such a database is stored in the memory, however, it is possible to obtain the objective variable (Y_{B}) without using the machine learning model.

The objective variable using the database is displayed specifically in the following manner.

That is, firstly, when the database of the molecular structures, the objective variables (Y_{C}), and the explanatory variables (X_{C}) of the organic compounds (C) used when the machine learning model is created is stored in the memory, judging whether or not an organic compound (C) having the explanatory variable (X_{C}) that exactly matches the explanatory variable (X_{B}) of the organic compound (B) exists in the database (Procedure G) after the explanatory variable is created (Procedure C).

Successively, when an organic compound (C) having an exactly matching explanatory variable exists in the database, reading the objective variable (Y_{C}) of the organic compound (C) having the exactly matching explanatory variable and displaying the objective variable (Y_{C}) as the objective variable (Y_{B}) in the VR space (Procedure H).

An objective variable (Y_{C}) stored in the database is a measured value or an estimated value calculated by a highly accurate molecular simulation method. The accuracy of Y_{C} therefore is usually higher than the accuracy of an objective variable (Y_{B}) estimated by using a machine learning model. It is possible to improve the estimation accuracy of the objective variable (Y_{B}) if not only the machine learning model but also the database is used.

### [1.5. Repetition of Molecular Structure Modification (Procedure E)]

After displaying the objective variable (Y_{B}) of the organic compound (B) in the VR space (after Procedure D or Procedure H), when the operator selects a new modification of the organic compound (B), repeating the Procedure B to Procedure D (Procedure E). Such repetition of molecular structure modification in the VR space can be done alone or performed in the state of simultaneous visualization for a large number of people.

### [1.6. Molecular Simulation (Procedure F)]

When the molecular structure is modified in the VR space and the organic compound (B) having an intended physical property value is found, it is also possible to apply molecular simulation to the found organic compound (B) and calculate the physical property value corresponding to the objective variable (Y_{B}) (Procedure F). Procedure F is not necessarily required but, if Procedure F is applied, it is possible to judge whether or not the newly found organic compound (B) meets a goal with a higher degree of accuracy.

### [2. Flowchart]

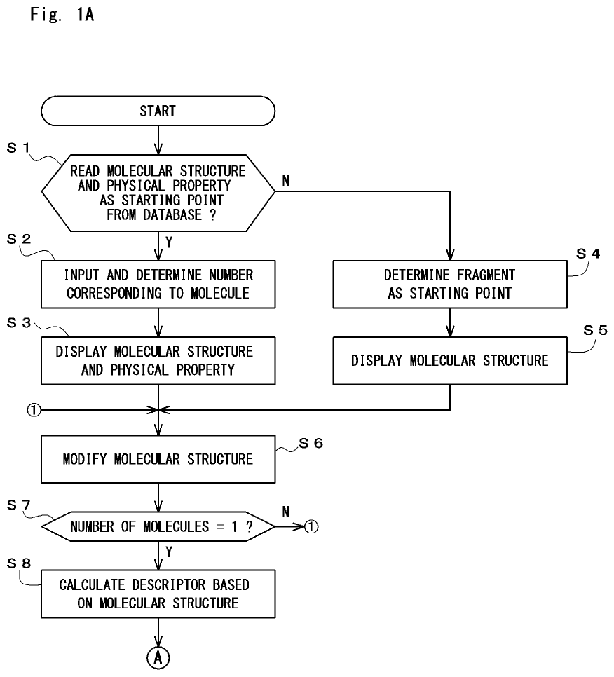

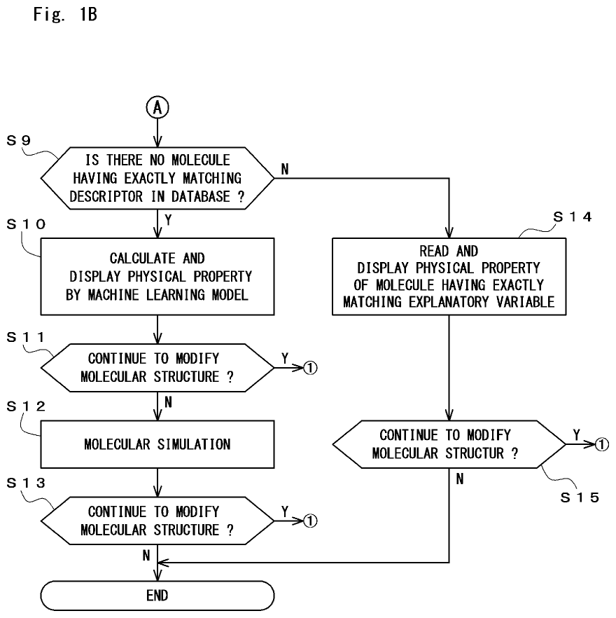

FIG. 1A shows a flowchart of a material design program according to the present invention. FIG. 1B shows a continuation of FIG. 1A.

### [2.1. Procedure A]

Firstly, requesting an operator to input a molecular structure of an organic compound (A) or a fragment as a starting point, displaying a stereoscopic image of the inputted molecular structure in a VR space, and simultaneously storing the inputted molecular structure in a memory (Procedure A).

When a database of molecular structures, objective variables (Y_{C}), and explanatory variables (X_{C}) of organic compounds (C) used when a machine learning model is created is stored in the memory, the process advances to Step 1 (hereunder also referred to merely as □S1□).

At S1, requesting the operator to select whether to read one of the organic compounds (C) stored in the database as the organic compound (A) from the database (Procedure A1). When to read from the database is selected (S1: YES), the process advances to S2.

At S2, requesting the operator to select an organic compound (C) to be read (Procedure A2). The method of selecting the organic compound (C) is not particularly limited. For example, when serial numbers are assigned to the organic compounds (C), it is possible to specify an organic compound (C) merely by inputting a number corresponding to the organic compound (C).

Successively, the process advances to S3. At S3, reading the molecular structure and the objective variable (Y_{C}) of the organic compound (C) selected by the operator from the database and displaying them as the molecular structure and the objective variable (Y_{A}) of the organic compound (A) in the VR space (Procedure A3). Simultaneously with this, storing the inputted molecular structure in the memory.

Here, when the database is not stored in the memory, S1 to S3 (Procedure A1 to Procedure A3) can be omitted.

Meanwhile, when to read from the database is not selected (S1: NO), the process advances to S4. At S4, requesting the operator to determine a fragment as a starting point (Procedure A4). Successively, at S5, displaying the structure of the determined fragment in the VR space (Procedure A5). Simultaneously with this, storing the determined molecular structure in the memory.

### [2.2. Procedure B]

After displaying a stereoscopic image of the molecular structure of the organic compound (A) or the fragment as the starting point in the VR space at S3 or S5, the process advances to S6. At S6, when the operator modifies the molecular structure of the organic compound (A) or the fragment and creates a new organic compound (B) in the VR space, displaying a stereoscopic image of the molecular structure of the organic compound (B) in the VR space and simultaneously storing the molecular structure of the organic compound (B) in the memory (Procedure B).

Successively, the process advances to S7. At S7, whether or not the number of molecules is 1 is judged. When a molecule is cleaved once or more at S6, two or more molecules will be displayed in the VR space. When multiple molecules exist in the VR space, it is not clear which molecule the objective variable (Y_{B}) should be estimated for. When the number of molecules in the VR space is not 1 (S7: NO) therefore, the process returns to S6 and requesting the operator to eliminate unnecessary molecules.

When the number of the molecules in the VR space is 1 (S7: YES) in contrast, the process advances to S8.

### [2.3. Procedure C]

At S8, creating an explanatory variable (X_{B}) that may correlate with an objective variable (Y_{B}), which is a physical property value of the organic compound (B), based on the molecular structure of the organic compound (B) and storing the explanatory variable (X_{B}) in the memory (Procedure C). The details of a method for creating the explanatory variable (X_{B}) are as described above and hence the explanations are omitted.

### [2.4. Procedure G]

Successively, the process advances to S9. At S9, when the database of the molecular structures, the objective variables (Y_{C}), and the explanatory variables (X_{C}) of the organic compounds (C) used when the machine learning model is created is stored in the memory, judging whether or not an organic compound (C) having the explanatory variable (X_{C}) that exactly matches the explanatory variable (X_{B}) of the organic compound (B) exists in the database (Procedure G). When such an organic compound (C) does not exist in the database (S9: YES), the process advances to S10. Here, as stated above, when such a database does not exist in the memory, S9 can be omitted.

### [2.5. Procedure D]

Successively, at S10, estimating the objective variable (Y_{B}) from the explanatory variable (X_{B}) by using a machine learning model in which the relationship between an objective variable (Y_{C}) of an organic compound (C) the molecular structure and physical property value of which are known and an explanatory variable (X_{C}) that may correlate with the objective variable (Y_{C}) has been learned beforehand, displaying the estimated objective variable (Y_{B}) in the VR space, and simultaneously storing the objective variable (Y_{B}) in the memory (Procedure D). The details of the machine learning model and the estimation of the objective variable (Y_{B}) using the machine learning model are as described above and hence the explanations are omitted.

### [2.6. Procedures E and F]

Successively, the process advances to S11. At S11, whether or not to continue the modification of the molecular structure is judged. When the operator selects a new modification of the organic compound (B) (S11: YES), the process returns to S6 and repeating the steps of S6 to S11 described above (Procedure E).

When the operator does not select a new modification of the organic compound (B) (S11: NO) in contrast, the process advances to S12. At S12, performing molecular simulation of the organic compound (B) and calculating a physical property value corresponding to the objective variable (Y_{B}) (Procedure F). As stated above, S12 is not necessarily required but the estimation accuracy of a physical property value improves if S12 is applied.

Successively, the process advances to S13. At S13, whether or not to continue the modification of the molecular structure is judged. When the operator selects a new modification of the organic compound (B) (S13: YES), the process returns to S6 and repeating the steps of S6 to S13 described above.

When the operator dose not select a new modification of the organic compound (B) (S13: NO) in contrast, the program ends.

### [2.7. Procedure H]

Meanwhile, when an organic compound (C) having an exactly matching explanatory variable exists in the database at S9 (S9: NO), the process advances to S14. At S14, reading the objective variable (Y_{C}) of the organic compound (C) having the exactly matching explanatory variable and displaying the objective variable (Y_{C}) as the objective variable (Y_{B}) in the VR space (Procedure H).

Successively, the process advances to S15. At S15, whether or not to continue the modification of the molecular structure is judged. When the operator selects a new modification of the organic compound (B) (S15: YES), the process returns to S6 and repeating the steps of S6 to S14 described above.

When the operator does not select a new modification of the organic compound (B) (S15: NO), the program ends.

### [3. Effect]

When a new organic compound (B) is created by modifying of a molecular structure in a VR space, an explanatory variable (for example, a type and a number of atoms, or a bonding state of atoms) that correlates with an objective variable (for example, viscosity) of the organic compound (B) is created. Successively, the objective variable is estimated from the created explanatory variable by using a previously learned machine learning model. Further, the estimated objective variable together with the molecular structure is displayed in the VR space.

When such operations are repeated in the VR space, it is possible to search a new organic compound having a physical property value suitable for an intended use. Further, when an operator is a person who has specialized knowledge on synthesis of an organic compound, it is possible to modify a molecular structure in consideration of factors such as □ease of synthesis and □cost□, which are hardly converted into data.

When MD calculation or experiment is used, it takes almost a few days to a few weeks before a physical property value of a designed organic compound is obtained and designer's thinking is temporarily stopped. In the present system in contrast, it is possible to estimate a physical property value in real-time (shorter than 1 second) by using the machine learning model. It is therefore possible to immediately seek out a new molecular structure without stopping designer's thinking. As a result, it is possible to inspire inspiration of a material designer on a new material and promote innovative new material design.

Additionally, although a high degree of computer skill is generally required for the use of machine learning, the present system can be operated with the feeling like using a molecular model and hence can be operated at a low degree of difficulty.

Moreover, by using Internet connection, it is possible to have a discussion while looking at the same molecule in the same VR space regardless of where this system is operated. Further, since the molecule can be observed from any position, inspiration can be given to more than one person.

### EXAMPLES

### [1. Creating Program]

The material design program shown in FIGS. 1A and 1B is created. In the program, the viscosity of a molecule is regarded as the objective variable and a structural descriptor is created with □RDKit□ based on the molecular structure. Further, the structural descriptor is regarded as the explanatory variable and a machine learning model is constructed by using □lightGBM□ that is a kind of machine learning method.

Further, a VR space where a molecular structure of an organic compound can be designed is programmed by using a game development platform □Unity□. Furthermore, an image in a VR space is projected to both eyes by using □Windows Mixed Reality headset□ of Acer Incorporated. In addition, the movement of both hands is captured by using □Leap Motion□ of Leap Motion Ltd.

### [2. Operation of Program]

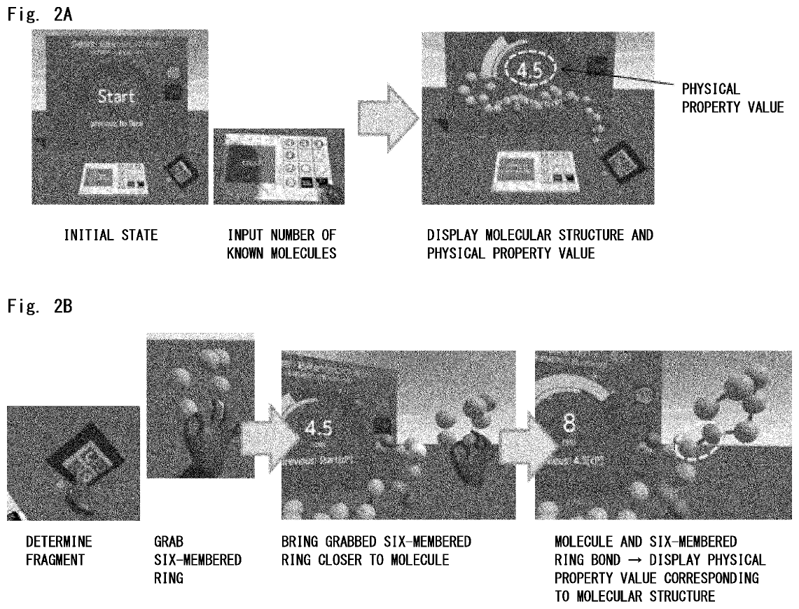

FIGS. 2 to 5 show actual display screens of the material design program.

### [2.1. Display of Organic Compound (A) as Starting Point]

A starting screen is shown in the left view of FIG. 2A. A numeric keypad is displayed at the lower part of the starting screen. When the molecular structure of a known molecule is stored in a memory, operating the numeric keypad in a VR space, and inputting the number assigned to the known molecule.

When the number is inputted, as shown in the right view of FIG. 2A, the molecular structure and the physical property value of an organic compound (A) corresponding to the number are displayed. In FIG. 2A, 4.5 is displayed as the physical property value.

### [2.2. Modification of Molecular Structure and Estimation of Objective Variable]

Successively, modification of the molecular structure is performed with a button or a hand gesture. Further, simultaneously with this, the estimation of the objective variable by using the machine learning model or reading of the objective variable from the database is performed.

### [2.2.1. Bond of Fragment]

When a six-membered ring is bonded to the molecule displayed on the screen for example, as shown in the left view of FIG. 2B, pressing the button corresponding to the six-membered ring in the buttons for selecting the fragments in the VR space, making the six-membered ring appear in the VR space, and grabbing the six-membered ring in the VR space.

Successively, as shown in the center view of FIG. 2B, bringing the grabbed six-membered ring closer to the molecule. As a result, as shown in the right view of FIG. 2B, the molecule and the six-membered ring bond and the molecular structure is modified. Almost simultaneously with this, the physical property value (the estimated value by the machine learning model or the physical property value stored in the memory) corresponding to the molecular structure is displayed in the VR space. In the FIG. 2B, it is shown that the physical property value increases from 4.5 to 8 by bonding the six-membered ring.

### [2.2.2. Replacement or Elimination of Atom]

When an atom is replaced or eliminated in the VR space, it is preferable to set rules in advance for finger operation in the VR space. Examples of such rules are

(a) an atom touched by the index finger of the right hand is replaced with a carbon atom,

(b) an atom touched by the two fingers of the index finger and the middle finger of the right hand is replaced with an oxygen atom, and

(c) when the middle finger and the ring finger of the right hand are folded, the right hand turns into a cane and, when an atom is touched with the cane, the atom is eliminated.

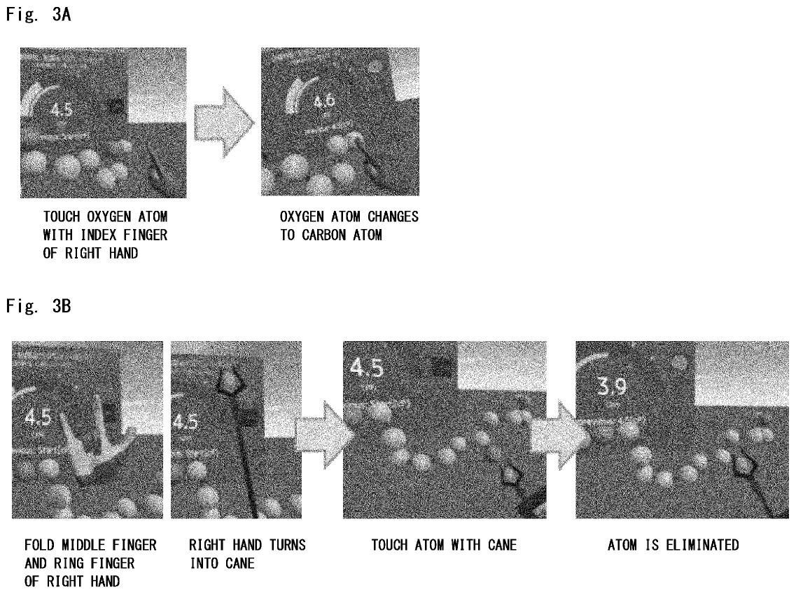

The replacement or elimination of an atom is performed concretely as follows. As shown in the left view of FIG. 3A for example, the index finger of the right hand is brought closer to an oxygen atom in a molecule. When the index finger of the right hand touches the oxygen atom, the oxygen atom is replaced with a carbon atom as shown in the right view of FIG. 3A. In the FIG. 3A, it is shown that the physical property value increases from 4.5 to 4.6 by the replacement from the oxygen atom to the carbon atom.

Otherwise, as shown in the left view of FIG. 3B, when the middle finger and the ring finger of the right hand are folded, the right hand turns into a cane. As a result, the function of eliminating an atom is activated. Successively, as shown in the center view of FIG. 3B, an atom is touched with the cane. As a result, as shown in the right view of FIG. 3B, the atom is eliminated and the molecular structure is modified. In FIG. 3B, it is shown that the physical property value reduces from 4.5 to 3.9 by the elimination of the atom.

### [2.2.3. Change, Cleavage, or Recombination of Bond]

When change, cleavage, or recombination of a bond is performed in the VR space, it is preferable to set rules in advance for finger operation in the VR space. Examples of such rules are

(a) a bond touched by the index finger of the left hand is changed to a single bond,

(b) a bond touched by the two fingers of the index finger and the middle finger of the left hand is changed to a double bond,

(c) when the middle finger and the ring finger of the left hand are folded, the left hand turns into scissors and, when a bond is touched with the scissors, the bond is cleaved, and

(d) when two atoms touched by the thumb of the right hand are brought closer to each other, the two atoms bond.

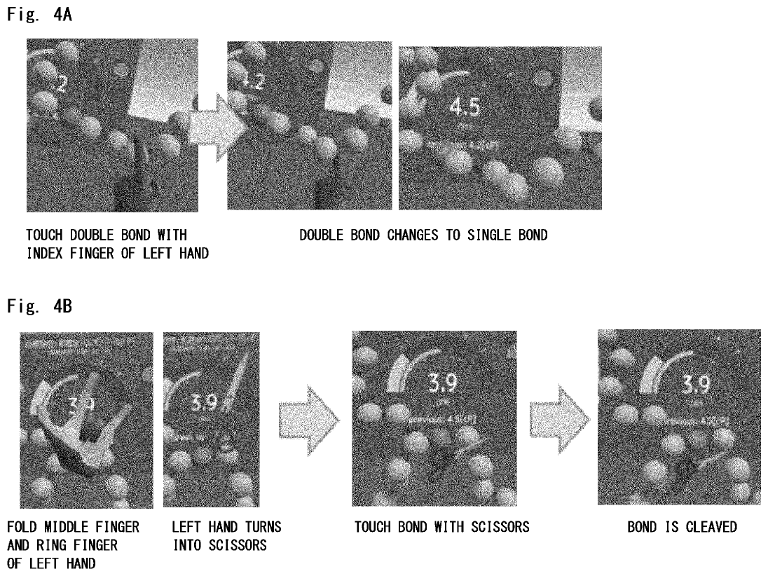

The change, cleavage, or recombination of a bond is performed concretely as follows. As shown in the left view of FIG. 4A for example, a double bond is touched with the index finger of the left hand. As a result, as shown in the right view of FIG. 4A, the double bond changes to a single bond. In FIG. 4A, it is shown that the physical property value increases from 4.2 to 4.5 by the change from the double bond to the single bond.

Otherwise, as shown in the left view of FIG. 4B, when the middle finger and the ring finger of the left hand are folded, the left hand turns to scissors. As a result, the function of cleaving a bond is activated. Successively, as shown in the center view of FIG. 4B, a bond is touched with the scissors.

As a result, as shown in the right view of FIG. 4B, the bond is cleaved. On this occasion, since two molecules are displayed in the VR space, the physical property value remains 3.9 and does not change. In order to display the physical property value of a cleaved molecule, eliminate the unnecessary molecule and set the number of molecules in the VR space to 1.

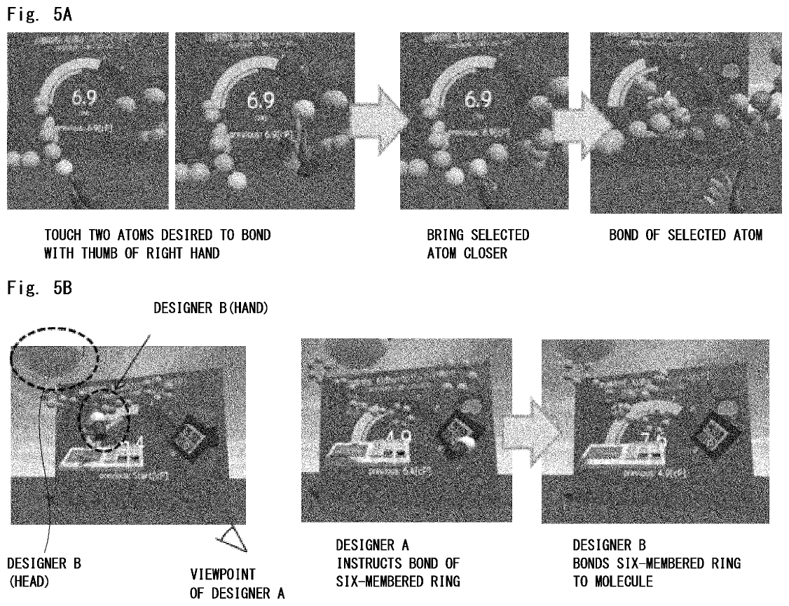

Otherwise, as shown in the left view of FIG. 5A, the two atoms included respectively in the two molecules desired to bond are touched with the thumb of the right hand. Successively, as shown in the center view of FIG. 5A, the selected two atoms are brought closer to each other. As a result, as shown in the right view of FIG. 5A, the selected two atoms bond and the molecular structure is modified. In FIG. 5A, it is shown that the physical property value reduces from 6.9 to 5.1 by the recombination of the molecules.

### [2.3. Multi-Person Discussion]

FIG. 5B shows the state of applying the material design program according to the present invention to multi-person discussion. The left view of FIG. 5B displays the head and hand of a designer B in the VR space and shows the state where a designer A sees the VR space. From the state, when the designer B is advised by the designer A to □bond a six-membered ring□ for example, as shown in the center view of FIG. 5B, the designer B makes the six-membered ring appear in the VR space. Successively, as shown in the right view of FIG. 5B, the designer B makes the six-membered ring bond to the molecule. Almost simultaneously with this, the physical property value increase from 4.9 to 7.6. In this way, by using the material design program according to the present invention, the modification of the molecular structure and the accompanying change of the physical property value can be visualized simultaneously for a large number of people.

Although the embodiments of the present invention have heretofore been explained in detail, the present invention is not limited to the embodiments at all and can be modified variously within a range not deviating from the tenor of the present invention.

The material design program according to the present invention can be used for new material creation, multi-person discussion, and others.

What is claimed is:

1. A material design program for making a computer perform the following procedures:
(A) Procedure A of requesting an operator to input a molecular structure of an organic compound (A) or a fragment as a starting point, displaying a stereoscopic image of the inputted molecular structure in a VR space, and simultaneously storing the inputted molecular structure in a memory;
(B) Procedure B of, when the operator modifies the molecular structure of the organic compound (A) or the fragment and creates a new organic compound (B) in the VR space, displaying a stereoscopic image of a molecular structure of the organic compound (B) in the VR space and simultaneously storing the molecular structure of the organic compound (B) in the memory;
(C) Procedure C of creating an explanatory variable (X_{B}) that may correlate with an objective variable (Y_{B}), which is a physical property value of the organic compound (B), based on the molecular structure of the organic compound (B) and storing the explanatory variable (X_{B}) in the memory;
(D) Procedure D of estimating the objective variable (Y_{B}) from the explanatory variable (X_{B}) by using a machine learning model in which the relationship between an objective variable (Y_{C}) of an organic compound (C) the molecular structure and physical property value of which are known and an explanatory variable (X_{C}) that may correlate with the objective variable (Y_{C}) has been learned beforehand, displaying the estimated objective variable (Y_{B}) in the VR space, and simultaneously storing the objective variable (Y_{B}) in the memory; and
(E) Procedure E of repeating Procedure B to Procedure D when the operator selects a new modification of the organic compound (B) after Procedure D.

2. The material design program according to claim 1,
wherein a database of the molecular structures, the objective variables (Y_{C}), and the explanatory variables (X_{C}) of the organic compounds (C) used when the machine learning model is created is stored in the memory; and
Procedure A includes
Procedure A1 of requesting the operator to select whether or not to read one of the organic compounds (C) stored in the database as the organic compound (A),
Procedure A2 of, when to read from the database is selected, requesting the operator to select an organic compound (C) to be read, and
Procedure A3 of reading the molecular structure and the objective variable (Y_{C}) of the organic compound (C) selected by the operator from the database and displaying them as the molecular structure and the objective variable (Y_{A}) of the organic compound (A) in the VR space.

3. The material design program according to claim 2, wherein, when to read from the database is not selected at Procedure A1, the material design program further includes:
Procedure A4 of requesting the operator to determine the fragment as the starting point; and
Procedure A5 of displaying the structure of the fragment in the VR space.

4. The material design program according to claim 1, wherein, when the operator does not select a new modification of the organic compound (B) in Procedure E, the material design program further includes Procedure F of performing molecular simulation of the organic compound (B) and calculating a physical property value corresponding to the objective variable (Y_{B}).

5. The material design program according to claim 1,
wherein a database of the molecular structures, the objective variables (Y_{C}), and the explanatory variables (X_{C}) of the organic compounds (C) used when the machine learning model is created is stored in the memory; and
the material design program further includes
Procedure G of judging whether or not an organic compound (C) having the explanatory variable (X_{C}) that exactly matches the explanatory variable (X_{B}) of the organic compound (B) exists in the database after Procedure C, and
Procedure H of, when an organic compound (C) having an exactly matching explanatory variable exists in the database, reading the objective variable (Y_{C}) of the organic compound (C) having the exactly matching explanatory variable and displaying the objective variable (Y_{C}) as the objective variable (Y_{B}) in the VR space.

6. The material design program according to claim 1, wherein the physical property value is viscosity.

7. The material design program according to claim 1, wherein Procedure B includes a procedure for modifying the molecular structure by using a button arranged in the VR space or a hand gesture.
